(19) United States
(12) Patent Application Publication
(10) Pub. No.: US 2015/0128934 A1
(43) Pub. Date: May 14, 2015

(54) WOUND TREATMENT AND CLEANING SYSTEM AND METHOD

(71) Applicant: Rapid Relief Solutions, Inc., Monterey, CA (US)

(72) Inventors: Howard S. SUTKIN, Los Gatos, CA (US); Theodore R. KUCKLICK, San Jose, CA (US); Charles R. BOGGS, Monterey, CA (US)

(73) Assignee: Rapid Relief Solutions, Inc., Monterey, CA (US)

(21) Appl. No.: 14/536,457

(22) Filed: Nov. 7, 2014

Related U.S. Application Data
(60) Provisional application No. 61/901,579, filed on Nov. 8, 2013.

(51) Int. Cl. A61M 19/00; B05B 11/00

(52) U.S. Cl. CPC A61M 19/00; B05B 11/3045
USPC 128/200.14; 29/428

(57) ABSTRACT

A wound treatment/cleaning device is described as having a sprayer portion and a brush portion each designed to be used with standard sized fluid bottles. The sprayer and brush can be packed with and/or sterilized with the fluid bottles, or packaged/sterilized separately. When the sprayer and brush are used in conjunction with the fluid bottle, the sprayer allows the fluid to be sprayed directly onto the wound to numb the wound while the brush can be used to clean the wound after it has been numbed.

### CROSS-REFERENCE TO RELATED APPLICATIONS

This application claims the benefit of priority to U.S. Provisional Application No. 61/901,579 filed Nov. 8, 2013, which is incorporated herein by reference in its entirety.

### FIELD OF THE INVENTION

The present invention relates to a system for use in the treatment of wounds, including abrasions and bums.

### BACKGROUND OF THE INVENTION

Skin wounds, such as “road rash” which are caused by severe and large scale skin abrasions, can be particularly dirty and unsanitary as a result of the nature of the injury. For example, if the road rash is caused by a motorcycle accident, the resulting wound may be large, bloody, painful, and likely embedded with gravel, dirt and other unsanitary debris. Because of the unsanitary nature of such wounds, cleaning of the wound is both necessary and painful.

Currently, the approach used to clean such wounds, when cleaning is approached, is to insert a hypodermic needle attached to a syringe into a bottle of diluted lidocaine HCl (lidocaine), withdraw on the plunger of the syringe to draw a volume of lidocaine into the syringe. The hypodermic needle/syringe is then removed from the bottle of lidocaine and the lidocaine is applied to the wound by pressing on the plunger of the syringe containing the lidocaine to expel a stream of lidocaine from the needle. This is performed with the needle tip near the wound so that the stream of lidocaine streaming from the needle is drizzled on the wound area to numb the area. The wound area is then cleaned using a brush, or sponge or similar.

The disadvantages to this approach are several. It is very difficult to drizzle the lidocaine on the entire wound using a syringe/needle. Areas of the wound may not be numbed and there is also a risk of further injury to the wound if the needle should contact the wound. The needle must be reinserted into the bottle of lidocaine numerous times risking injury and contamination. Also, depending on who is treating the wound, the components, lidocaine, needle, syringe, brush and/or other cleaning supplies, are not likely to be located together, even in an emergency vehicle, such as an ambulance. Locating the various components needed to treat the wound delays treatment of the wound which can increase the chances of complications and infection.

### BRIEF SUMMARY OF THE INVENTION

The wound treatment and cleaning system and method described here solves the problems associated with the current approach to cleaning wounds. The treatment/cleaning system described here enables the responding medical personnel, emergency room personnel, or even the victim or nonmedical personnel with the victim to rapidly and easily anesthetize, clean and prepare the wound for dressing.

The wound treatment/cleaning device includes a sprayer portion and a brush portion. The sprayer and brush are designed to be used with standard sized fluid bottles. The sprayer and brush can be packed with and/or sterilized with the fluid bottles, or packaged/sterilized separately. When used in conjunction with the fluid bottle, the sprayer allows the fluid to be sprayed directly onto the wound to numb the wound. When used in conjunction with the fluid bottle, the brush can be used to clean the wound after it has been numbed.

In another embodiment, the wound treatment system may generally comprise a sprayer body having a pump and a sprayer cap configured for positioning over, upon, or against a cap of a fluid bottle, wherein the pump defines a sprayer opening positioned transversely relative to the pump. The system may further include a rigid hollow tubular body having a tapered piercing tip, wherein the tubular body extends from the sprayer body and is in fluid communication with the pump. Additionally, the system may further include a brush cap configured for positioning over, upon or against a body of the fluid bottle.

In yet another embodiment, the system may be packaged as a kit for wound treatment which may generally comprise a sprayer body having a pump and a sprayer cap configured for positioning over, upon, or against a cap of a fluid bottle, wherein the pump defines a sprayer opening positioned transversely relative to the pump. The kit may further include a rigid hollow tubular body having a tapered piercing tip, wherein the tubular body extends from the sprayer body and is in fluid communication with the pump. Additionally, the kit may further include a brush cap configured for positioning over, upon or against a body of the fluid bottle, wherein the brush cap comprises a plurality of bristles extending from the brush cap.

In one embodiment for assembling the wound treatment assembly, the method may generally comprise inserting a rigid hollow tubular body having a tapered piercing tip through a seal positioned on a fluid bottle such that the piercing tip is inserted within the fluid bottle while maintaining sterility within the fluid bottle, and positioning a sprayer body having a pump and a sprayer cap over, upon, or against a cap of the fluid bottle, wherein the pump defines a sprayer opening positioned transversely relative to the pinup and wherein the tubular body extends from the sprayer body and is in fluid communication with the pump. Such a method may further include securing a brush cap over, upon or against a body of the fluid bottle, wherein the brush cap comprises a plurality of bristles extending from the brush cap.

### BRIEF DESCRIPTION OF THE DRAWINGS

FIG. 1 shows the components of the wound treatment and cleaning system.

FIG. 2 shows the components of the wound treatment and cleaning system as they are being assembled.

FIG. 3 shows the components of the wound treatment and cleaning system after assembly.

FIG. 4 shows an alternative embodiment of the wound treatment and cleaning system.

FIG. 5 shows an alternative embodiment of the wound treatment and cleaning system.

### DETAILED DESCRIPTION OF THE INVENTION

In an emergency situation, as in the case of a motorcycle or bicycle accident, it is crucial to treat and clean large abrasive injuries as quickly as possible. The wound treatment/cleaning system described here allows for this type of treatment/cleaning by emergency response personnel, by nonemergency response personnel, and even by the wound victim himself, or non-medical people with the victim. Because the wound treatment and cleaning system can be used with standard sized bottles of cleaning and/or numbing fluids, such as lidocaine, it is very flexible and portable and can be packaged as a kit which can be easily carried by either emergency response personnel, or others.

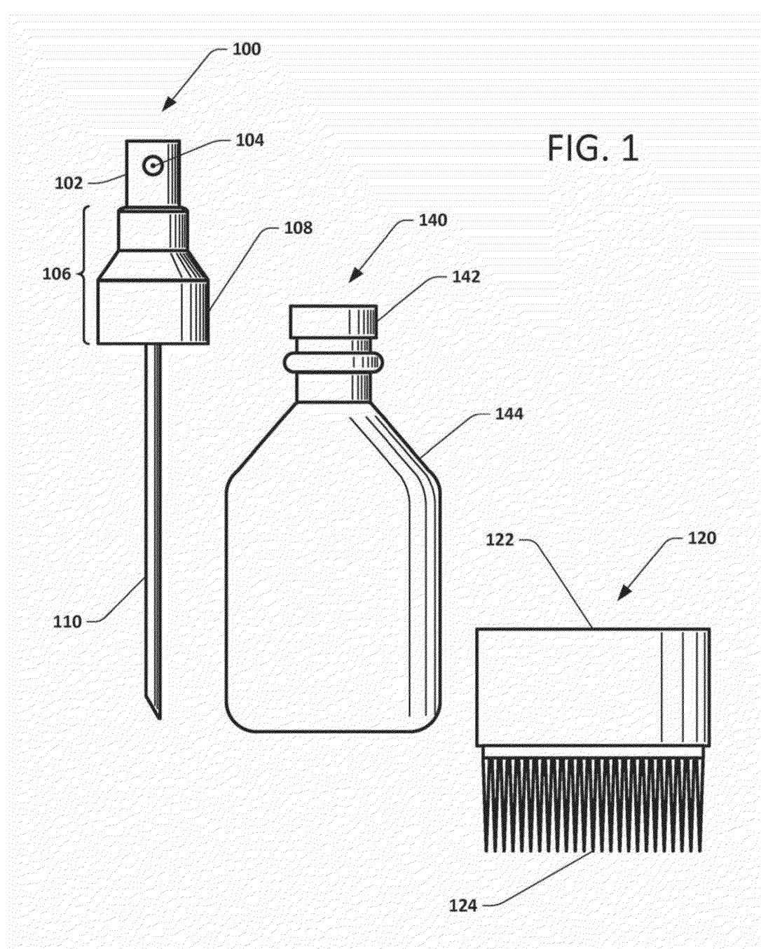

FIG. 1 shows the components of one embodiment of the wound treatment and cleaning system as well as a standard sized bottle of fluid, such as lidocaine. Sprayer 100 and brush 120 make up the wound treatment and cleaning system that is designed to be used with fluid bottle 140. Sprayer 100 includes a sprayer pump 102, a sprayer opening 104, a sprayer body 106, a sprayer cap 108 and a hollow tubular body, or hollow piercer, or needle 110 having a tapered piercing tip.

Brush 120 includes a brush cap 122 and brush bristles 124.

Bottle 140 includes a bottle cap 142 and a bottle body 144.

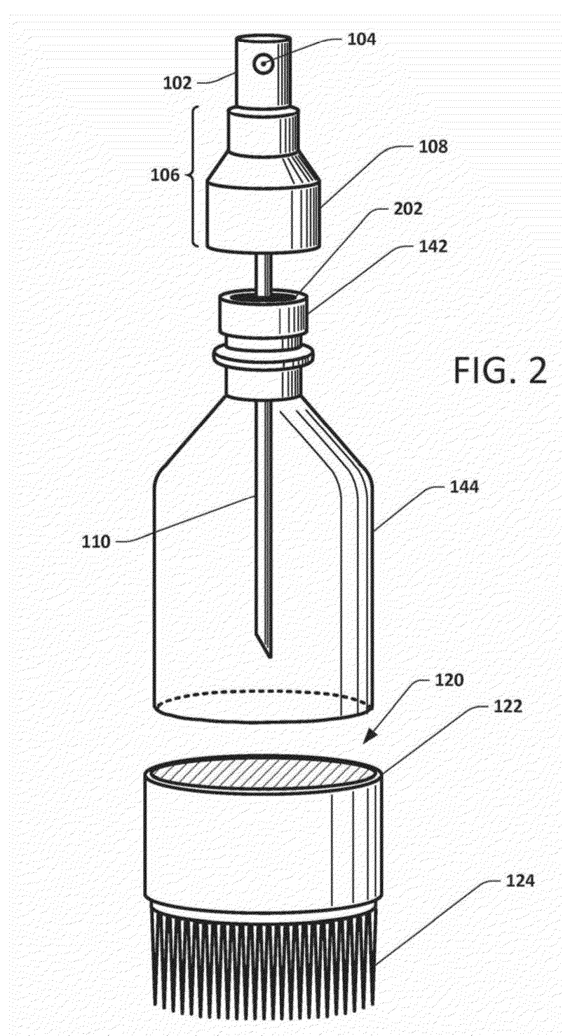

FIG. 2 shows the components of an embodiment of the wound treatment and cleaning system and how they fit with bottle 140. Bottle cap 142 may be a standard sterile bottle cap which includes rubberized center portion 202. The rubberized center portion allows needles to be punctured through the cap of the bottle while maintaining a fluid tight seal and sterility.

Sprayer needle 110 is designed to puncture through cap 142 of the fluid bottle and sprayer cap 108 is designed to fit over, upon, or against cap 142 of the fluid bottle. To engage the sprayer with the fluid bottle, the sprayer needle is punctured through the rubberized center portion of the bottle cap, and placed within the body of the bottle and in contact with any fluid in the bottle. The needle is pushed through the bottle cap until the sprayer cap is snuggly fitted over the bottle cap.

Sprayer pump 102 is designed to slide up and down within sprayer body 106. After the sprayer is engaged with a fluid bottle, this pumping motion causes the fluid from the fluid bottle to spray through the sprayer opening 104. After it is engaged with the bottle, the sprayer pump can be used to pump fluid out of the bottle by repeatedly pressing down on the sprayer pump while holding the bottle. The snug fit of the sprayer cap over the bottle cap stabilizes the sprayer and bottle so that it can be used easily with one hand.

In other embodiments, different variations of a sprayer pump may be used. For instance, rather than utilizing a sprayer pump 102, a pressurized cartridge may be inserted into the bottle to provide for a pressurized sprayer. Moreover, different variations of the sprayer pump 102 may also be incorporated to provide for alternative fluid ejection configurations from the bottle such as spray, stream, etc., depending upon the desired configuration of fluid ejection.

If the sprayer component of the wound treatment and cleaning system is sterile, then the insertion of the needle through the cap of the bottle allows sterile fluid to be sprayed on the wound. If the fluid is lidocaine, this serves to numb the wound for subsequent wound cleaning.

Brush cap 122 of brush 120 is designed to fit over body 144 of the bottle such that the brush cap 122 forms at least a partially cylindrical body defining, an opening into which the bottom portion of the body 144 of the bottle may be received. The inner dimensions of brush cap 122 are such that the brush cap fits snugly over, upon, or against the body of the bottle. Once the brush is engaged with the bottle in this way, brush bristles 124 can be used to clean the wound after the wound has been numbed with the fluid using the sprayer. The brush bristles 124, as shown, may extend or project distally from the lower surface of the brush cap 122. However, the brush bristles 124 may optionally and/or additionally also extend or project along one or more sides of the brush cap 122 to provide for different brush surfaces which for wound cleaning. Additionally, while the brush bristles 124 are shown to project uniformly from the lower surface of the brush cap 122, the brush bristles 124 may be formed into any number of different shapes or contours for facilitating wound cleaning along different regions of the body. The bottle helps to add stability to the brush making it easier to use.

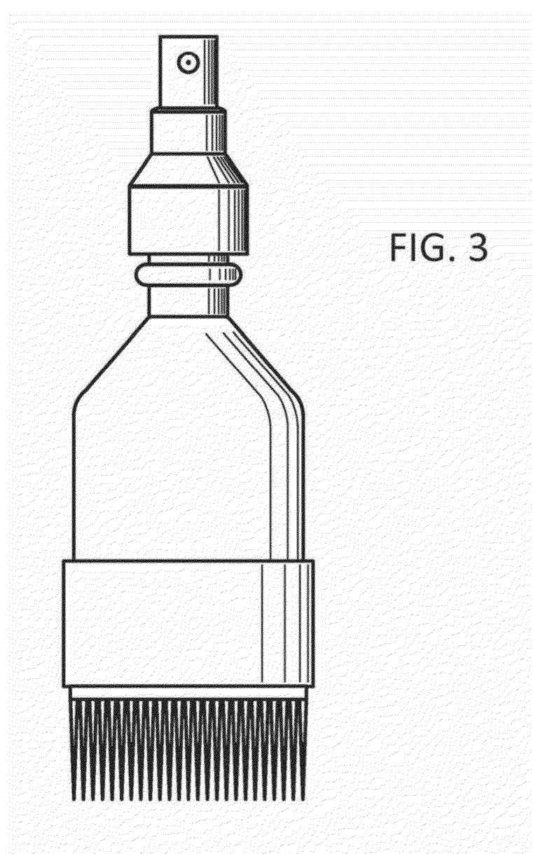

FIG. 3 shows an embodiment of the wound treatment and cleaning system sprayer and wound treatment and cleaning system brush fully engaged with the fluid bottle. In this configuration both the sprayer and the bush can be easily used with one hand to numb and clean the wound.

In a preferred embodiment of wound treatment and cleaning system bottle 140 is a standard lidocaine bottle. The diameter of the cap portion of the bottle preferably is about 1.8-2.2 cm, but may be smaller or larger. The diameter of the rubberized center of the cap preferably is about 0.6-1.0 cm, but may be smaller or larger. The diameter of the body of the bottle is preferably about 3.8-4.2 cm, but may be smaller or larger. The height of the bottle including the body and the cap is preferably about 7.6-8.4 cm, but may be smaller or larger. The bottle is preferably made from a relatively rigid material which can be sterilized such as a polymer or glass. The rigidity of the material helps stabilize the wound treatment and cleaning system so that it can be used easily with one hand.

While one preferred embodiment of bottle 140 is described, other embodiments and sizes of the bottle 140 may be used with the features described herein. For instance, relatively larger or smaller bottles may be used and the sprayer 100 and brush 120 may be sized accordingly to accommodate different sized bottles.

In a preferred embodiment the fluid in the bottle is sterile and is not under pressure.

In a preferred embodiment the bottle is rigid enough so that it cannot be squeezed significantly by human hand pressure.

In a preferred embodiment sprayer pump 102 and body 106 are made of a rigid material which preferably can be sterilized such as a polymer or metal. Needle 110 is preferably made of a rigid material which can be sterilized and which is preferably rigid enough to be made strong, hollow and with a pointed end such as metal or a polymer. The outer diameter of the needle preferably is about 1-5 mm, but may be smaller or larger. The length of the needle preferably is about 7.0-8.0 cm, but may be shorter or longer.

In a preferred embodiment inner diameter of sprayer cap 108 is about 1.75-2.15 cm, but may be smaller or larger and is designed to snuggly fit over bottle cap 142. The diameter of the inside of the sprayer cap may gradually decrease from the opening inward so that it can accommodate a wider range of bottle cap diameters. The inside of the sprayer cap is preferably non-threaded so that the fit between the sprayer cap and the bottle cap is a press fit. The inside of the sprayer cap may have a softer inner surface, such as one of silicone, which provides a snug fit with the bottle cap.

The dimensions described herein are exemplary of some preferred embodiments. However, alternative dimensions may also be incorporated.

In a preferred embodiment sprayer pump moves parallel to sprayer body 106 when pressed. Preferably the resulting spray of the fluid which emanates from sprayer opening 104 sprays in a direction which is approximately perpendicular to the motion of the sprayer pump.

In a preferred embodiment the brush cap 122 is made from a rigid material which preferably can be sterilized such as a polymer or metal. The material of brush bristles 124 is preferably flexible enough so that when formed into bristles, the material acts as a stiff brush that can effectively clean a wound, but with enough flexibility to clean the wound comfortably. Preferably, the bristle material is sterilizable such as a polymer. The inside of the brush cap may have a softer inner surface, such as one of silicone, which provides a snug fit with the bottle body. However, in other embodiments, various biocompatible materials may be used for the bristle material.

In a preferred embodiment the inner diameter of the bush cap is about 3.75-4.15 cm, but may be smaller or larger and is designed to snuggly fit over bottle body 144. The diameter of the inside of the brush cap may gradually decrease from the opening inward so that it can accommodate a wider range of bottle body diameters. The inside of the brush cap is preferably non-threaded so that the fit between the brush cap and the bottle body is a press fit.

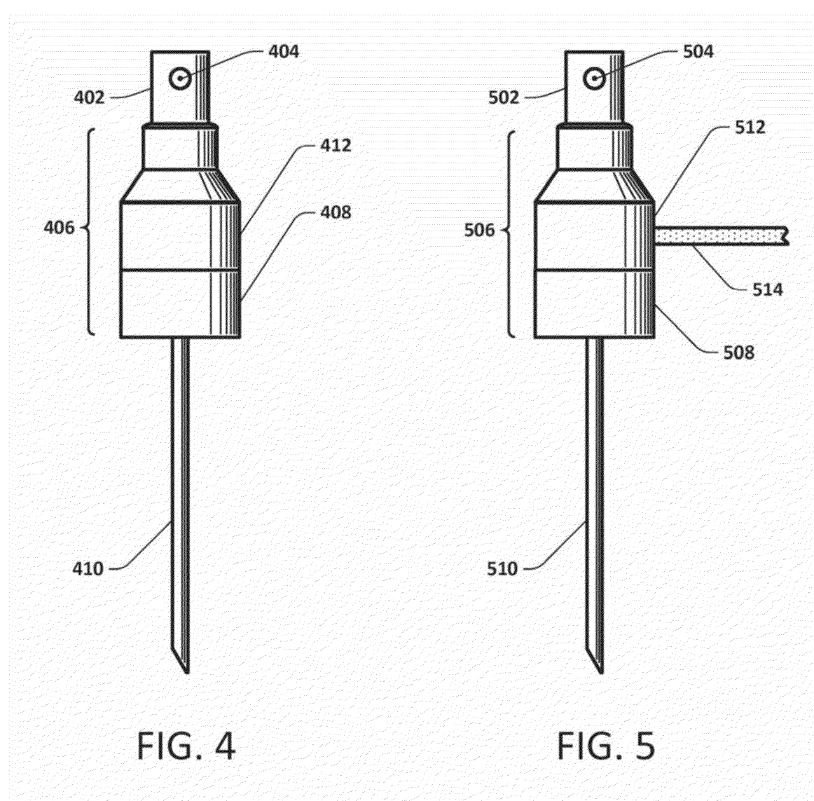

FIG. 4 shows the sprayer of another embodiment of the wound treatment and cleaning system. In this embodiment, a fluid and another substance are mixed and applied to the wound. Sprayer needle 410 is designed to puncture through the cap of the fluid bottle and sprayer cap 408 is designed to fit over the cap of the fluid bottle. The needle is pushed through the bottle cap until the sprayer cap is snuggly fitted over the bottle cap.

Sprayer pump 402 is designed to slide up and down within sprayer body 406. After the sprayer is engaged with a fluid bottle, this pumping motion causes the fluid from the fluid bottle to spray through sprayer opening 404. After it is engaged with the bottle, the sprayer pump can be used to pump fluid out of the bottle by repeatedly pressing down on the sprayer pump while holding the bottle. The snug tit of the sprayer cap over the bottle cap stabilizes the sprayer and bottle so that it can be used easily with one hand. Reservoir 412 may contain a powder, a gas or a second fluid which is to be mixed with the first fluid in the fluid bottle. Activating sprayer pump 402 causes the substance in reservoir 412 to mix with the fluid in the fluid bottle and as a result, a mixture of the fluid and the substance is sprayed out of sprayer opening 404.

FIG. 5 shows the sprayer of another embodiment of the wound treatment and cleaning system. In this embodiment, a fluid and another substance are mixed and applied to the wound. Sprayer needle 510 is designed to puncture through the cap of the fluid bottle and sprayer cap 508 is designed to fit over the cap of the fluid bottle. The needle is pushed through the bottle cap until the sprayer cap is snuggly fitted over the bottle cap.

Sprayer pump 502 is designed to slide up and down within sprayer body 506. After the sprayer is engaged with a fluid bottle, this pumping motion causes the fluid from the fluid bottle to spray through sprayer opening 504. After it is engaged with the bottle, the sprayer pump can be used to pump fluid out of the bottle by repeatedly pressing down on the sprayer pump while holding the bottle. The snug fit of the sprayer cap over the bottle cap stabilizes the sprayer and bottle so that it can be used easily with one hand. Reservoir 512 may contain a powder, a gas or a second fluid which is to be mixed with the first fluid in the fluid bottle. Reservoir 512 may be fed by connector 514 which leads to a larger reservoir containing the second substance. Activating sprayer pump 502 causes the substance in reservoir 512 to mix with the fluid in the fluid bottle and as a result, a mixture of the fluid and the substance is sprayed out of sprayer opening 504.

In some embodiments the fluid in the bottle is lidocaine and the substance in reservoir 412 or reservoir 512 is sodium bicarbonate.

In some embodiments the fluid in the bottle is a mixture of lidocaine and sodium bicarbonate.

In some embodiments one fluid is sprayed from the wound treatment and cleaning system. In some embodiments a mixture of one fluid with another substance is sprayed from the wound treatment and cleaning system. In some embodiments 2 substances are mixed within, and sprayed from, the wound treatment and cleaning system. In some embodiments more than 2 substances are mixed within, and sprayed from, the wound treatment and cleaning system.

Polymers used in the wound treatment and cleaning system may include polypropylene, polyethylene, silicone, polyester, nylon, or any other suitable polymer. Metals include stainless steel, titanium, cobalt-chromium, Nitinol, or any other suitable metal. These materials are only meant to be examples and it will be understood that the components of the wound treatment and cleaning system may be made out of any suitable material.

It should be appreciated that although certain embodiments have been presented here, other variations are envisioned. For example, the sprayer cap and/or the brush cap may be threaded, or may have a snap feature to connect them to the bottle. The sprayer pump may be in different configurations such as a trigger pump or aerosol spray.

The applications of the disclosed invention discussed above are not limited to certain embodiments but may include any number of other variations. Modification of the above-described methods and devices for carrying out the invention and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A wound treatment system, comprising:
a sprayer body having a pump and a sprayer cap configured for positioning over, upon, or against a cap of a fluid bottle, wherein the pump defines a sprayer opening positioned transversely relative to the pump; and
a rigid hollow tubular body having a tapered piercing tip, wherein the tubular body extends from the sprayer body and is in fluid communication with the pump.

2. The system of claim 1 further comprising a brush cap configured for positioning over, upon or against a body of the fluid bottle.

3. The system of claim 2 wherein the brush cap comprises a plurality of bristles extending from the brush cap.

4. The system of claim 2 wherein the brush cap is configured to press-fit over, upon, or against the body of the fluid bottle.

5. The system of claim 1 wherein the sprayer cap is configured to press-fit over, upon, or against the cap of the fluid bottle.

6. The system of claim 1 further comprising a reservoir in fluid communication with the pump.

7. The system of claim 1 wherein the fluid bottle comprises a reservoir of lidocaine.

8. A kit for wound treatment, comprising:
a sprayer body having a pump and a sprayer cap configured for positioning over, upon, or against to cap of a fluid bottle, wherein the pump defines a sprayer opening positioned transversely relative to the pump; and
a rigid hollow tubular body having a tapered piercing tip, wherein the tubular body extends from the sprayer body and is in fluid communication with the pump.

9. The kit of claim 8 further comprising a brush cap configured for positioning over, upon or against a body of the fluid bottle, wherein the brush cap comprises a plurality of bristles extending from the brush cap.

10. The kit of claim 8 further comprising the fluid bottle.

11. The kit of claim 10 wherein the fluid bottle comprises a reservoir of lidocaine.

12. The kit of claim 8 wherein the brush cap is configured to press-fit over, upon, or against the body of the fluid bottle.

13. The kit of claim 8 wherein the sprayer cap is configured to press-fit over, upon, or against the cap of the fluid bottle.

14. The kit of claim 8 further comprising a reservoir in fluid communication with the pump.

15. A method of assembling a wound treatment assembly, comprising:
inserting a rigid hollow tubular body having a tapered piercing tip through a seal positioned on a fluid bottle such that the piercing tip is inserted within the fluid bottle while maintaining sterility within the fluid bottle; and
positioning a sprayer body having a pump and a sprayer cap over, upon, or against a cap of the fluid bottle, wherein the pump defines a sprayer opening positioned transversely relative to the pump and wherein the tubular body extends from the sprayer body and is in fluid communication with the pump.

16. The method of claim 15 further comprising securing a brush cap over, upon or against a body of the fluid bottle, wherein the brush cap comprises a plurality of bristles extending from the brush cap.

17. The method of claim 15 further comprising actuating the pump such that a fluid within the fluid bottle is drawn through the tubular body and out through the sprayer opening in a direction transverse relative to the pump.

18. The method of claim 17 further comprising actuating the pump repeatedly.

19. The method of claim 17 further comprising actuating the pump in a single-handed operation.
